(19) United States
(12) Patent Application Publication
(10) Pub. No.: US 2013/0130992 A1
(43) Pub. Date: May 23, 2013

(54) DIPEPTIDE DERIVATIVE FOR THE TREATMENT OF CANCER

(71) Applicants: Sunil Kumar Jaiwant Rao Deshmukh, Mumbai (IN); Shilpa Amit Verekar, Mumbai (IN); Prabhu Dutt Mishra, Mumbai (IN); Sreekumar Sankaranarayanan Eyyammadichiyil, Mumbai (IN); Kalpana Sanjay Joshi, Mumbai (IN); Heinz-Herbert Fiebig, Freiburg (DE); Gerhard Kelter, Ehrenkirchen (DE)

(72) Inventors: Sunil Kumar Jaiwant Rao Deshmukh, Mumbai (IN); Shilpa Amit Verekar, Mumbai (IN); Prabhu Dutt Mishra, Mumbai (IN); Sreekumar Sankaranarayanan Eyyammadichiyil, Mumbai (IN); Kalpana Sanjay Joshi, Mumbai (IN); Heinz-Herbert Fiebig, Freiburg (DE); Gerhard Kelter, Ehrenkirchen (DE)

(73) Assignees: ONCOTEST GMBH, Freiburg, (DE); PIRAMAL ENTERPRISES LIMITED, Mumbai (IN)

(21) Appl. No.: 13/813,716

(22) PCT Filed: Aug. 5, 2011
(86) PCT No.: PCT/IB2011/053502

§ 371 Date: Feb. 1, 2013

Related U.S. Application Data
(60) Provisional application No. 61/371,904, filed on Aug. 9, 2010.

(51) Int. Cl. C07K 5/072

(52) U.S. Cl. CPC C07K 5/06104
USPC 514/19.4; 540/456; 514/21.1; 514/19.3; 514/19.5; 514/19.6; 435/119

(57) ABSTRACT

This invention relates to purified compound of formula (1). The invention includes all isomeric forms and all tautomeric forms of the compound of formula (1) and pharmaceutically acceptable salts thereof. The present invention further relates to processes for the production of the compound of formula (1) by fermentation of the fungal strain of sterile mycelium (PM0509732/MTCC5544) and to pharmaceutical compositions containing the compound as active ingredient and its use in medicines for treatment of cancer.

### FIELD OF THE INVENTION

This invention relates to a compound of formula (1) having anticancer activity. The compound may be obtained by fermentation of a microorganism belonging to endophytic fungal strain (PM0509732/MTCC5544). The invention also includes all isomeric forms and all tautomeric forms of the compound of formula (1) and pharmaceutically acceptable salts thereof. The present invention further relates to processes for the production of the anticancer compound, to the microorganism belonging to endophytic fungal strain (PM0509732/MTCC5544) and to pharmaceutical compositions containing the compound as active ingredient and their use in medicines for treatment of cancer.

### BACKGROUND OF THE INVENTION

Cancer is a generic term for a large group of diseases caused by uncontrolled growth and spread of cells that can affect any part of the body. Other terms used for cancer are malignant tumors and neoplasms. Cancer is a leading cause of death worldwide. As per the World Health Organization (WHO) report the most frequent types of cancer are lung, stomach, liver, colorectal, oesophagus and prostate in men, and breast, lung, stomach, colorectal and cervical in women.

The type of treatment and therapies used are largely determined by the location of the cancer in the body and also the extent to which the cancer has spread at the time of diagnosis. Cancer therapy currently falls under the following categories including surgery, radiation therapy, chemotherapy, bone marrow transplantation, stem cell transplantation, hormonal therapy, immunotherapy, antiangiogenic therapy, targeted therapy, gene therapy and others.

There are reports of anticancer compounds such as Taxol, Vincristine, Torreyanic acid, and Camptothecin from natural resources. (Natural Product Communications, 2009, Vol. 4 (11), 1513). In spite of this, there is still a need for drugs which can effectively treat various cancers such as lung, prostate, pancreatic and head and neck cancer.

### SUMMARY OF THE INVENTION

The present invention relates to a compound designated herein as compound of formula (1).

The present invention also relates to a purified compound, (designated herein as compound of formula (1)), isolated from the fermented broth of the microorganism belonging to endophytic fungal strain (PM0509732/MTCC5544).

The invention also relates to all isomeric forms and tautomeric forms of compound of formula (1), and pharmaceutically acceptable salts thereof.

The compound of formula (1), isomers, tautomers, or pharmaceutically acceptable salts thereof, are useful for the treatment of cancer.

The invention further relates to pharmaceutical compositions comprising the compound of formula (1), an isomer, a tautomer, a pharmaceutically acceptable salt thereof, as an active ingredient in association with a pharmaceutically acceptable carrier or vehicle for the treatment of cancer.

The present invention further relates to processes for the production of the compound of formula (1) and/or its isomers or its tautomers from the microorganism belonging to endophytic fungal strain (PM0509732/MTCC5544).

The present invention also relates to processes for the isolation of the microorganism belonging to endophytic fungal strain (PM0509732/MTCC5544), which on cultivation produces the compound of formula (1), its isomers and its tautomers.

### BRIEF DESCRIPTION OF THE DRAWING

FIG. 1: Illustrates ¹HNMR (DMSO-d₆; 500 MHz; Instrument: Bruker) of the compound of formula (1).

### DETAILED DESCRIPTION OF THE INVENTION

The compound of formula (1) has the molecular formula C₁₈H₃₀N₂O₅S₂ (molecular weight 418). The compound of formula (1) may be characterised by any one or more of the physico-chemical and spectral properties, such as high performance liquid chromatography (HPLC), mass spectrum (MS), infra red (IR) and nuclear magnetic resonance (NMR) spectroscopic data as discussed herein below.

The compound of formula (1) is structurally represented as follows:

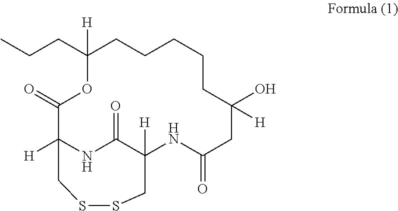 [SMILES: CCCC1CCCCCC(O)CC(=O)NC2CSSCC(NC2=O)C(=O)O1]

The microorganism, which may be used for the production of the compound of formula (1), is an endophytic fungal strain of sterile mycelium (PM0509732/MTCC5544), herein after referred to as culture no. PM0509732, which is isolated from the leaves of Pongamia pinnata (family Fabaceae) collected from Karnala Bird Sanctuary near Panvel in Raigad District, Maharashtra, India.

The present invention further provides processes for the production of the compound of formula (1) from culture no. PM0509732 comprising the steps of:
(a) cultivating the culture no. (PM0509732/MTCC5544) or one of its variants or mutants under submerged aerobic conditions in nutrient medium containing one or more sources of carbon and one or more sources of nitrogen and nutrient inorganic salts;
(b) isolating the compound of formula (1) from the culture broth; and
(c) purifying the compound of formula (1).

The step (c) involving purification of the compounds of formula (1) is carried out by purification procedures generally used in the related art.

The compound of formula (1) produced according to the process of the present invention is a substantially pure compound. Thus, the compound of formula 1 is an isolated pure compound having anticancer activity.

As used herein, the term “mutant” refers to an organism or cell carrying a mutation, which is an alternative phenotype to the wild-type.

As used herein, the term “variant” refers to an individual organism that is recognizably different from an arbitrary standard type in that species.

The term “mammal” as used herein, refers to a human as well as non-human mammals, including but not limited to, cows, horses, pigs, dogs and cats. The term “mammal” may be used interchangeably with the term “patient” or “subject”.

The term “active ingredient” as used herein, refers to the compound of formula (1) or to a isomer or tautomer or a pharmaceutically acceptable salt thereof.

The term “substantially pure” as used herein, means that the compound of formula (1) and isomers thereof are sufficiently pure such that further purification would not detectably alter the physical and chemical properties, such as enzymatic and biological activities, of the substance. Compounds of formula (1) can be purified substantially by following the methods known to those skilled in the art.

The term “compound of formula (1)” includes compound of formula (1) and isomers, tautomers, and pharmaceutically acceptable salts thereof.

As used herein the term “therapeutically effective amount” in reference to the treatment of cancer (as listed herein) using the compound of formula (1) refers to an amount capable of invoking one or more of the following effects in a subject receiving the compound of the present invention: (i) inhibition, to some extent, of tumor growth, including, slowing down and complete growth arrest; (ii) reduction in the number of tumor cells; (iii) reduction in tumor size; (iv) inhibition (i.e., reduction, slowing down or complete stopping) of tumor cell infiltration into peripheral organs; (v) inhibition (i.e., reduction, slowing down or complete stopping) of metastasis; (vi) enhancement of anti-tumor immune response, which may, but does not have to, result in the regression of the tumor; and/or (vii) relief, to some extent, of one or more symptoms associated with the cancer being treated.

The term “pharmaceutically acceptable salt(s)”, as used herein, means those salts of compounds of the invention that are safe and effective in mammals and that possess the desired biological activity. Pharmaceutically acceptable acid addition salts include, but are not limited to, hydrochloride, hydrobromide, hydroiodide, nitrate, sulfate, phosphate, acetate, lactate, salicylate, citrate, tartrate, ascorbate, succinate, maleate, fumarate, formate, benzoate, glutamate, methanesulfonate, benzensulfonate, or p-toluenesulfonate salts. Suitable base addition salts include, but are not limited to, calcium, lithium, magnesium, potassium, sodium, or zinc, salts.

Preliminary identification of culture no. PM0509732, which is the producer of compound of formula (1), was performed by examination of its colony characteristics. Microscopic studies on the strain of isolated culture no. PM0509732 were carried out on Potato dextrose agar medium. The observations were made after 4, 7, 14, 21 and 30 days of incubation at 25° C. Culture no. PM0509732 has been identified as an endophytic fungal strain of sterile mycelium.

Growth on Potato dextrose agar medium develops as 64 mm diameter colonies in 5 days at 26° C. The colony is initially white then turned yellowish green (Jaune curry RAL 1027). The back/reverse surface is initially white then turned to grey brown (RAL 8019) after 30 days of incubation. Formation of neither pigmentation nor droplet is observed.

Culture no. PM0509732 has been deposited with Microbial Type Culture Collection (MTCC), Institute of Microbial Technology, Sector 39-A, Chandigarh-160 036, India, a World Intellectual Property Organization (WIPO) recognized International Depository Authority (IDA) and has been given the accession number MTCC5544.

In addition to the specific microorganism described herein, it should be understood that mutants of PM0509732, such as those produced by the use of chemical or physical mutagens including X-rays, U.V. rays etc. and organisms whose genetic makeup has been modified by molecular biology techniques, may also be cultivated to produce the compound of formula (1).

The screening for suitable mutants and variants which can produce the compound according to the invention can be confirmed by HPLC, NMR, IR, MS determination of biological activity of the active compounds accumulated in the culture broth, for example by testing the compounds for anticancer activity or by a combination thereof.

The medium and/or nutrient medium used for isolation and cultivation of culture no. PM0509732, which produces the compound of formula (1), preferably contains sources of carbon, nitrogen and nutrient inorganic salts. The carbon sources are, for example, one or more of starch, glucose, sucrose, dextrin, fructose, molasses, glycerol, lactose, or galactose. A preferred carbon source is soluble starch and glucose. The sources of nitrogen are, for example, one or more of soyabean meal, peanut meal, yeast extract, beef extract, peptone, malt extract, corn steep liquor, gelatin, or casamino acids. Preferred nitrogen source is soyabean meal and yeast extract. The nutrient inorganic salts are, for example, one or more of sodium chloride, potassium chloride, calcium chloride, manganese chloride, magnesium chloride, strontium chloride, cobalt chloride, potassium bromide, sodium fluoride, sodium hydrogen phosphate, potassium hydrogen phosphate, dipotassium hydrogen phosphate, disodium phosphate, calcium carbonate, sodium bicarbonate, sodium silicate, sodium nitrate, ammonium nitrate, potassium nitrate, sodium sulphate, ammonium sulphate, ammonium heptamolybdate, ferric citrate, copper sulphate, magnesium sulphate, ferrous sulphate, zinc sulphate or boric acid. Calcium chloride, sodium chloride and sodium nitrate are preferred.

The maintenance of culture no. PM0509732 may be carried out at a temperature ranging from 21° C. to 35° C. and a pH of about 6.5 to 8.5. Typically, culture no. PM0509732 is maintained at 26° C.-29° C. and a pH of about 6.5. The well-grown cultures may be preserved in the refrigerator at 6° C.-12° C.

Seed culture cultivation of culture no. PM0509732 may be carried out at a temperature ranging from 24° C. to 31° C. and a pH of about 5.5 to 8.5, for 100-125 hours at 180-220 rpm (revolutions per minute). Typically, culture no. PM0509732 seed is cultivated at 25° C.-27° C. and a pH of about 5.5, for 115-120 hours at 180-200 rpm.

The production of the compound of formula (1) may be carried out by cultivating culture no PM0509732 by fermentation at a temperature ranging from 25° C. to 31° C. and a pH of about 5.5 to 8.5, for 60-100 hours at 180-220 rpm. Typically, culture no. PM0509732 is cultivated at 26° C.-31° C. and pH 5.5-7.7 for 66-96 hours at 190-210 rpm.

The production of the compound of formula (1) can be carried out by cultivating culture no. PM0509732 in a suitable nutrient broth under conditions described herein, preferably under submerged aerobic conditions, for example in shake flasks. The progress of fermentation and production of the compound of formula (1) can be detected by high performance liquid chromatography (HPLC) and by measuring the bioactivity of the culture broth by testing against the cancer cell lines.

Fermentation is a process of growing microorganisms for the production of various chemical or pharmaceutical compounds. Microbes are normally incubated under specific conditions in the presence of nutrients.

In the resulting culture broth, the compound of formula (1) is present in the culture filtrate as well as in cell mass and can be isolated using different extraction methods and chromatographic techniques. Thus, the compound of formula (1) can be recovered from the culture filtrate by extraction with a water immiscible solvent such as petroleum ether, dichloromethane, chloroform, ethyl acetate, diethyl ether or butanol, or by hydrophobic interaction chromatography using polymeric resins such as “Diaion HP-20®” (Mitsubishi Chemical Industries Limited, Japan), “Amberlite XAD®” (Rohm and Haas Industries, USA) or adsorption on activated charcoal. These techniques may be used repeatedly, alone or in combination. The compound of formula (1) can be recovered from the cell mass by extraction with a water miscible solvent such as methanol, acetone, acetonitrile, n-propanol, or iso-propanol or with a water immiscible solvent such as petroleum ether, dichloromethane, chloroform, ethyl acetate or butanol. One other option is to extract the whole broth with a solvent selected from petroleum ether, dichloromethane, chloroform, ethyl acetate, methanol, acetone, acetonitrile, n-propanol, iso-propanol, or butanol. Typically, the compound of formula (1) is extracted from the cell mass using acetone and by chromatography using Diaion HP-20 from the culture filtrate. Concentration and lyophilization of the extracts gives the active crude material.

The compound of formula (1) of the present invention can be recovered from the crude material by fractionation using any of the following techniques: normal phase chromatography (using alumina or silica gel as stationary phase; eluents such as petroleum ether, ethyl acetate, dichloromethane, acetone, chloroform, methanol, or combinations thereof; and additions of amines such as NEt₃); reverse phase chromatography (using reverse phase silica gel such as dimethyloctadecylsilylsilica gel, (RP-18) or dimethyloctylsilyl silica gel (RP-8) as stationary phase; and eluents such as water, buffers (for example, phosphate, acetate, citrate (pH 2-8)), and organic solvents (for example methanol, acetonitrile, acetone, tetrahydrofuran, or combinations of these solvents)); gel permeation chromatography (using resins such as Sephadex LH-20® (Pharmacia Chemical Industries, Sweden), TSKgel® Toyopearl HW (TosoHaas, Tosoh Corporation, Japan) in solvents such as methanol, chloroform, acetone, ethyl acetate, or their combinations); or by counter-current chromatography (using a biphasic eluent system made up of two or more solvents such as water, methanol, ethanol, iso-propanol, n-propanol, tetrahydrofuran, acetone, acetonitrile, methylene chloride, chloroform, ethyl acetate, petroleum ether, benzene, and toluene). These techniques may be used repeatedly, alone or in combination. A typical method is chromatography over normal phase silica gel and reverse phase silica gel (RP-18).

As used herein, the term “isomer” is a general term used for all isomers of the compound of formula (1) that differ only in the orientation of their atoms in space. The term isomer includes mirror image isomers (enantiomers), mixtures of mirror image isomers (racemates, racemic mixtures) and isomers of compounds with more than one chiral center that are not mirror images of one another (diastereoisomers). The compound of the present invention may have asymmetric centers and occur as racemates, racemic mixtures, individual diastereoisomers, or enantiomers, or may exist as geometric isomers, with all isomeric forms of said compound being included in the present invention.

As used herein, the term “tautomer” refers to the coexistence of two (or more) compounds that differ from each other only in the position of one (or more) mobile atoms and in electron distribution, for example, keto-enol tautomers.

Compound of formula (1), exists as an equilibrium mixture of two compounds which may be isomers or tautomers.

The compound of formula (1), isomers and tautomers thereof can be converted into their pharmaceutically acceptable salts which are all contemplated by the present invention.

The salts can be prepared by standard procedures known to one skilled in the art, for example, salts like sodium and potassium salts, can be prepared by treating the compound of formula (1), isomers, and tautomers thereof, with a suitable sodium or potassium base, for example sodium hydroxide, potassium hydroxide. Similarly, salts like hydrochloride and sulphate salts, can be prepared by treating the compound of formula (1), isomers, and tautomers thereof, with a suitable acid, for example hydrochloric acid, and sulphuric acid.

The compound of formula (1) has anticancer activity against a wide range of cancer cells.

The compound of formula (1), isomers, tautomers or pharmaceutically acceptable salts thereof, can be administered to animals, such as mammals, including humans, as pharmaceuticals and in the form of a pharmaceutical composition. The compound of formula (1), isomers, tautomers, pharmaceutically acceptable salts thereof, can be administered to a patient who is diagnosed with cancer.

Accordingly, the present invention also relates to the compound of formula (1), isomers, tautomers or pharmaceutically acceptable salts thereof for use as pharmaceuticals and to the use of the compound of formula (1), isomers, tautomers or pharmaceutically acceptable salts thereof for the production of medicaments having anticancer activity.

The present invention further relates to pharmaceutical composition, which contain an effective amount of the compound of formula (1) and/or isomers and/or tautomers and/or one or more pharmaceutically acceptable salts thereof, together with a pharmaceutically acceptable carrier. The effective amount of the compound of formula (1), or its stereoisomer, or its tautomer or its pharmaceutically acceptable salt as the active ingredient in the pharmaceutical preparations normally is from about 0.01 mg to 1000 mg.

The present invention also relates to a method for the manufacture of a medicament containing the compound of formula (1) and/or isomers and/or tautomers and/or one or more pharmaceutically acceptable salts thereof, for the treatment of cancer.

The compounds of the present invention are particularly useful as anti-cancer agents. The present invention accordingly relates to the use of the compound of formula (1) and/or isomers and/or tautomers and/or one or more pharmaceutically acceptable salts thereof, for the manufacture of a medicament for the treatment of cancer.

The compounds of the present invention i.e. the compound of formula (1) and/or isomers and/or tautomers and/or one or more pharmaceutically acceptable salts find use in the treatment of cancers. Compounds of the present invention are used to reduce, inhibit, or diminish the proliferation of tumor cells, and thereby assist in reducing the size of a tumor. Representative cancers that may be treated by such compounds include but are not limited to bladder cancer, breast cancer, colorectal cancer, endometrial cancer, gastric cancer, head & neck cancer, kidney cancer, melanoma, non-small-cell lung cancer, ovarian cancer, pancreatic cancer, prostate cancer, renal cancer, soft tissue sarcoma, oesophageal cancer, cancer of uterus, testicular and germ cell cancer, thyroid cancer, cervix cancer, pleuramesothelioma, brain tumors (glioblastoma, cerebellar astrocytoma, cerebral astrocytoma, ependymoma, medulloblastoma, neuroblastoma, retinoblastoma, supratentorial primitive neuroectodermal and pineal tumors, visual pathway and hypothalamic glioma, brain stem glioma), liver cancer, Ewing's sarcoma family of tumors, osteosarcoma, malignant fibrous histiocytoma of bone, rhabdomyosarcoma, skin cancer, small-cell lung cancer, Wilms' tumors, leukemias (acute lymphoblastic leukemia, adult acute myeloid leukemia, chronic lymphocytic leukemia, chronic myeloid leukemia), and lymphomas (Hodgkin's disease, non-Hodgkin's lymphoma, hairy cell leukemia, multiple myeloma, primary central nervous system lymphoma), among others.

According to an embodiment, the present invention provides a method for the treatment of cancer by administering to a mammal in need thereof a therapeutically effective amount of a compound of formula (1).

The compound of the present invention can be administered orally, nasally, topically, subcutaneously, intramuscularly, intravenously, or by other modes of administration.

Pharmaceutical compositions which contain compound of formula (1) or isomers or tautomers or a pharmaceutically acceptable salt thereof, an be prepared by mixing the compound of formula (1), with one or more pharmacologically tolerated auxiliaries and/or excipients such as, wetting agents, solubilisers such as surfactants, vehicles, tonicity agents, fillers, colorants, masking flavors, lubricants, disintegrants, diluents, binders, plasticizers, emulsifiers, ointment bases, emollients, thickening agents, polymers, lipids, oils, cosolvents, complexation agents, or buffer substances, and converting the mixture into a suitable pharmaceutical form such as, for example, tablets, coated tablets, capsules, granules, powders, creams, ointments, gels, syrup, emulsions, suspensions, or solutions suitable for parenteral administration.

Examples of auxiliaries and/or excipients that may be mentioned for use in preparation of pharmaceutical composition are cremophor, poloxamer, benzalkonium chloride, sodium lauryl sulphate, dextrose, glycerin, magnesium stearate, polyethylene glycol, starch, dextrin, lactose, cellulose, carboxymethylcellulose sodium, talc, agar-agar, mineral oil, animal oil, vegetable oil, organic and mineral waxes, paraffin, gels, propylene glycol, benzyl alcohol, dimethylacetamide, ethanol, polyglycols, Tween 80, solutol HS 15, and water. It is also possible to administer the active ingredient as such, without vehicles or diluents, in a suitable form, for example, in capsules.

As is customary, the galenic formulation and the method of administration as well as the dosage range which are suitable in a specific case depend on the species to be treated and on the state of the respective condition or disease, and can be optimized using methods known in the art. On average, the daily dose of active compound in a patient is 0.05 mg to 100 mg per kg, typically 1 mg to 50 mg per kg. If required, higher or lower daily doses can also be administered. Actual dosage levels of the active ingredient in the pharmaceutical compositions of this invention may be varied so as to obtain an amount of the active ingredient, which is effective to achieve the desired therapeutic response for a particular patient, composition, and mode of administration without resulting in unacceptable side effects to the patient.

The following are provided as illustrative examples of the present invention and do not limit the scope thereof.

### EXAMPLES

The following terms/symbol/abbreviations/chemical formulae are employed in the examples:
l: Litre
ml: Millilitre
kg: Kilogram
g: Gram
mg: Milligram
mm: Millimeter
cm: Centimeter
μ: Micron
nm: Nanometer
hrs: Hours
min: Minute
mg/l: Milligram per litre
mg/ml: Milligram per Millilitre
μg/ml: Microgram per Millilitre
μl: Microlitre
ml/min: Millilitre per Minute
mM: Millimole
μM: Micromole
rpm: Revolutions per Minute
λ: Wavelength
IC₅₀: 50% Inhibitory concentration
ATCC: American Type Culture Collection
NCI: National Cancer Institute
DSMZ: Deutsche Sammlung von. Mikroorganismen and Zellkulturen
GmbH: (German Collection of Microorganisms and Cell Cultures)
PAA: PAA Laboratories GmbH
RPMI: Roswell Park Memorial Institute
v/v: Volume (of solute) per volume (of solvent).
PCR: Polymerase Chain Reaction
DNA: Deoxy ribose Nucleic Acid
NSCLC: Non-Small Cell Lung Carcinoma
DMSO: Dimethyl sulphoxide

### Example 1

### Isolation of Culture No. PM0509732 Collected from Plant Source

a) Composition of the Isolation Medium:

Potato dextrose agar (PDA) media (Hi Media, Mumbai, India) supplemented with 50 mg/l chloramphenicol was used for isolation purpose. While preparing the medium chloramphenicol was added after dissolving the same in 10 ml of 95% ethanol and mixing thoroughly with the hot medium. The medium was autoclaved at 121° C. for 15 min The pH prior to autoclaving was adjusted to 6.5 (at 25° C.).

b) Procedure

The leaves of the plant Pongamia pinnata (family Fabaceae) were collected from Karnala Bird Sanctuary near Panvel in Raigad District, Maharashtra, India in the month of March 2005. These samples were collected in polythene bags and brought to the laboratory and the fresh leaves of the samples were processed for isolation of fungi.

The undamaged fresh leaves of plant Pongamia pinnata were washed thoroughly with water, air dried, surface sterilized by treating with 4% sodium hypochlorite (NaOCl, Qualigens) in water for 1 min and subsequently washed with sterilized demineralised water to remove traces of the disinfectants. These were cut into segments of about 5 mm with sterile surgical blade. Five segments of leaves were aseptically placed equidistant on 90 mm Petri plate containing above media (Potato dextrose agar) so that they were separated from each other by 2-3 cm. The plates were incubated in alternative 12 hrs dark/light chamber at 26° C.+1° C. After 6 days an elongating hypha over the medium was picked up at its tips with thin needle, and it was transferred to new medium for purification purpose. The isolates were repeatedly sub-cultured on to Potato dextrose agar slants to get pure culture no. PM0509732. The slants were incubated for 14 days at 26° C.

### Example 2

### Purification of Culture No. PM0509732

a) Composition of the Isolation Medium:

Potato dextrose agar (PDA) media (Hi Media, Mumbai, India) supplemented with 50 mg/l chloramphenicol was used for isolation purpose. While preparing the medium chloramphenicol was added after dissolving the same in 10 ml of 95% ethanol and mixing thoroughly with the hot medium. The medium was autoclaved at 121° C. for 15 min The pH prior to autoclaving was adjusted to 6.5 (at 25° C.).

b) Procedure:

The culture was available on Potato dextrose agar medium in 90 mm diameter Petri plate. Another plate with Potato dextrose agar medium was inoculated with the growing mycelial tips of the culture under binocular microscope. As and when the growth was observed on the plate it was then transferred to fresh slant. The slants were incubated for 10 days at 25° C. These were then used for shake flask fermentation.

### Example 3

### Maintenance of Producer Strain—Culture No. PM0509732

a) Composition of isolation medium

Potato dextrose agar (PDA) media (Hi Media, Mumbai, India) supplemented with 50 mg/l chloramphenicol was used for isolation purpose. While preparing the medium chloramphenicol was added after dissolving the same in 10 ml of 95% ethanol and mixing thoroughly with the hot medium. The pH prior to autoclaving was adjusted to 6.5. After dissolving the ingredients thoroughly by heating, the resultant solution was distributed in test tubes and sterilized by autoclaving at 121° C. for 15 min. The test tubes with media were allowed to solidify in a slanting position.

The agar slants were inoculated with the growing culture of PM0509732 aseptically by a wire loop and incubated at 26-29° C. until a good growth was observed. The well-grown cultures were stored in the refrigerator at 6-12° C.

### Example 4

### Fermentation of the Culture No. PM0509732 in Shake Flasks

a) Composition of seed culture medium:

Soluble Starch 15.0 g; Soya Bean Meal 15.0 g; Glucose 5.0 g; Calcium Chloride (CaCl₂) 2.0 g; Sodium Chloride (NaCl) 5.0 g; Yeast Extract 2.0 g; Corn Steep Liquor 1.0 g; Glycerol 11.6 g; Ammonium Heptamolybdate ((NH₄)₆Mo₇O₂₄.4H₂O) 0.16 mg; Zinc Sulphate (ZnSO₄.7H₂O) 0.22 mg; Manganese Chloride (MnCl₂.4H₂O) 0.5 mg; Ferrous Sulphate (FeSO₄.7H₂O) 0.5 mg; Copper Sulphate (CuSO₄.5H₂O) 0.16 mg; Cobalt Chloride (CoCl₂.6H₂O) 0.16 mg; Demineralised Water 1.01, pH 5.5 prior to autoclaving.

b) The above medium was distributed in 100 ml amount in 500 ml Erlenmeyer flasks and autoclaved at 121° C. for 20 min. The flasks were cooled and then inoculated with the above mentioned (Example 3) well producing strain (culture no. PM0509732) on the slant and fermented on shaker at 200 rpm for 120 hrs at 26° C.±1° C. to give the seed. This was used as the seed culture for inoculating the production with the following composition:

c) Composition of the production medium:

Glucose 30.0 g; Sodium Nitrate (NaNO₃) 3.0 g; Dipotassium Hydrogen Phosphate (K₂HPO₄) 1.0 g; Potassium Chloride (KCl) 0.5 g; Magnesium Sulphate (MgSO₄.7H₂O) 0.5 g; Ferrous Sulphate (FeSO₄.7H₂O) 0.01 g; Demineralized Water 1.0 l, pH 5.5 prior to autoclaving.

d) The production medium was distributed in 200 ml in each 1000 ml Erlenmeyer flask and autoclaved at 121° C. for 20 min The flasks were cooled to 25-28° C. and then inoculated with the seed culture (1% v/v) obtained in step (b).

e) Fermentation parameters

Temperature 26-30° C.; agitation 190-210 rpm; harvest time 66-96 hrs.

The progress of fermentation and production of the compound of formula (1) can be detected by high performance liquid chromatography (HPLC) and by measuring the bioactivity of the culture broth by testing against the cancer cell lines.

### Example 5

### Isolation and Characterization of Compound of Formula (1)

20.0 l of whole broth was filtered to separate biomass and culture filtrate. The biomass (1.0 kg) was sequentially extracted using 7.0 l acetone followed by 2.0 l of methanol, and filtered and concentrated. The residue obtained was suspended in water (1.0 l) and, extracted using petroleum ether followed by chloroform. Organic layer was concentrated under reduced pressure to get crude extract.

The crude extract was subjected to silica gel column chromatography. The active compound was eluted using step gradient mode with chloroform and methanol mixture. The final purification was performed on preparative RP-18 silica gel column chromatography.
Preparative HPLC conditions:
Column: Eurospher RP-18 (250 mm×20 mm), 10μ
Flow rate: 20 ml/min
Detection: UV 220 nm
Solvent system: An isocratic run of acetonitrile and water (40:60) for 16 min, followed by additional 4 min gradient of 40-100% acetonitrile in water.

The active compounds were eluted between 11-16 min and the solvents evaporated to obtain a white powder which was analyzed by analytical HPLC.
Analytical HPLC conditions:
Column: Lichrospher RP-18 (125 mm×4.6 mm), 5μ
Flow rate: 0.9 ml/min
Solvent system: Acetonitrile and water (45:55)
Detection: UV 220 nm
Retention time: 8.01 min (T1) & 9.15 min (T2).

The sample contains two compounds T1 & T2, and it has been observed that even after separation, these compounds exist as a mixture. This was confirmed by analytical HPLC. The peaks T1 and T2 were collected separately as two fractions and dried under vacuum and reinjected. Both the fractions eluted again as two peaks with retention time 8.01 (T1) and 9.15 (T2). This was further indicated by LCMS and UV data of the compounds.

Culture filtrate (18 l) separated from whole broth was passed through a column of HP-20 resin. The column was washed with water and eluted using methanol (2 l). Active methanol eluate was concentrated to obtain crude extract. The extract was suspended in water. The aqueous suspension was extracted using pet ether followed by chloroform. Organic layer was concentrated and crude extract was obtained, which contain compounds T1 and T2 (analysed by HPLC).

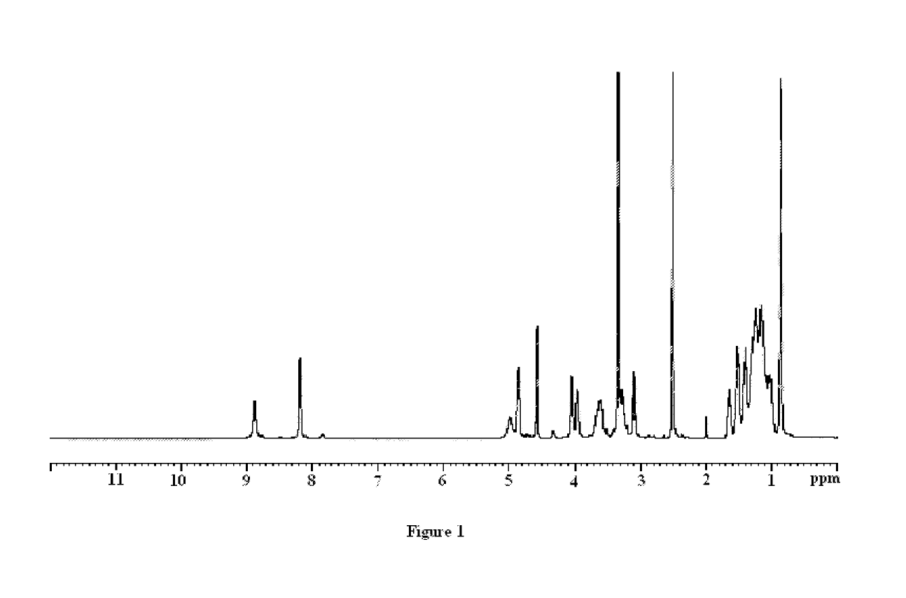

Physical and Spectral Properties:

| | |
|---|---|
| Appearance | White powder |
| Solubility | Soluble in pyridine, acetic acid, dimethyl sulfoxide; |
| | sparingly soluble in chloroform and methanol; and |
| | insoluble in water |
| UV | Detected by HPLC - PDA in acetonitrile and water at |
| | 220 nm for both the peaks |
| Mass | ESI LCMS: T1 418.9 (M + H), T2 418.9 (M + H) |
| | ESI HRMS: 419.1640 (M + H), 441.1466 (M + Na) |
| Molecular | C₁₈H₃₀N₂O₅S₂ |
| formula | |
| Elemental | Obtained: C 51.80%, H 6.71%, N 6.46% and S 15.33%; |
| analysis | Calculated for C₁₈H₃₀N₂O₅S₂: C 51.67%, H 7.17%, |
| | N 6.69% and S 15.31% |
| IR (KBr) | 3345, 1646, 1671, 1715, 1459, 1399 cm⁻¹ |
| ¹H NMR | δ 8.8 (bs, 1H), 8.1 (bs, 1H), 4.9 (bs, 1H), 4.8 (bs, 1H), |
| (500 MHz, | 4.5 (bs, 1H), 4.0 (s, 1H), 3.9 (s, 1H), 3.6 (m, 2H), 3.2 |
| DMSO-d₆) | (m, 1H), 3.1 (d, 1H), 1.6-1.0 (m, 16H, methylene groups), |
| | 0.8 (t, 3H) (as given in FIG. 1) (bs refers to broad |
| | singlet) |
| ¹³C NMR | δ 174.17, 173.91, 169.42, 74.50, 70.30, 52.07, 51.26, |
| (75 MHz, | 43.25, 42.10, 36.20, 35.60, 34.30, 32.90, 27.90, 26.70, |
| DMSO-d₆) | 22.00, 18.30 and 13.70 |

### Biological Evaluation of the Compound of Formula (1)

### Example 6

### In-Vitro Assays

### Monolayer Assay

### Step 1

### Maintenance of the Cell Lines

Oncotest GmbH, Germany's proprietary tumor cell line panel comprises 40 cell lines. These cell lines were derived from 15 different tumor histotypes, each represented by 1 to 6 different cell lines. Cell lines were established from cancer of the bladder (3), colon (4), head and neck (1), lung (6), breast (3), pancreas (3), prostate (4), ovary (2), kidney (3), liver (1), stomach (1) and the uteri body (1), as well as from melanoma (3), sarcoma (2) and pleuramesothelioma (3). Out of these 40 cell lines, 24 cell lines were established at Oncotest, from patient-derived tumor xenografts. The other 16 cell lines were either provided by the NCl (Bethesda; MD, USA) or were purchased from ATCC (Rockville, Md., USA) or DSMZ (Braunschweig, Germany).

Cell lines were routinely passaged once or twice weekly and maintained in culture for upto 20 passages. All the cells were grown at 37° C. in a humidified atmosphere with 5% (Carbon Dioxide) CO₂ in RPMI 1640 medium supplemented with 10% (v/v) fetal calf serum (FCS) and 0.1 mg/ml gentamicin (medium and all other components from PAA, Colbe, Germany). Authenticity of all cell lines was proven by STR (short tandem repeat) analysis, a PCR based DNA-fingerprinting methodology.

### Step 2

### Sample Preparation

Stock solutions of the compound of formula (1) (3.3 mM in DMSO) were prepared and stored in small aliquots (200 μl) at −20° C. An aliquot of the stock solution was thawed on the day of use and stored at room temperature prior to and during dosing. The subsequent dilutions were done with complete RPMI 1640 cell culture medium as follows: the stock solution was diluted 1:22 followed by serial dilutions finally resulting in a (compared to the test concentration) 15-fold higher concentration. For the final dilution step (1:15), 10 μl/well of the solution was directly added to 140 μl/well culture medium. Compound of formula (1) [also referred as test compound] was tested at 0.00032, 0.001, 0.0032, 0.01, 0.032, 0.1, 0.32, 1, 3.2 and 10 μM. Each concentration was evaluated in triplicate.

### Step 3

### Assay

A modified Propidium Iodide assay was used to assess the effect of the compound of formula (1) on the growth of the human tumor cell lines and was designed as in reference, Anti-cancer Drugs, 6, 522-532, (1995), the disclosure of which is incorporated by reference for the teaching of the assay.

Cells were plated in 96-well flat-bottom microtiter plates at a cell density of 4,000 to 20,000 cells/well. After a 24 hr recovery period to allow the cells to resume exponential growth, the compound of formula (1) was applied at 10 concentrations in half-log increments in triplicates and treatment continued for 4 days. After 4 days of treatment, cells were next washed with 200 μl phosphate buffer solution (PBS) to remove dead cells, then 200 μl of a solution containing 7 μg/ml propidium iodide (PI) and 0.1% (v/v) Triton X-100 were added to the wells. After an incubation period of 1-2 hours at room temperature, fluorescence (FU) was measured using the Cytofluor 4000 microplate reader (excitation λ=530 nm, emission λ=620 nm) to quantify the amount of attached viable cells. Pharmacological effects on cell proliferation and survival were expressed as Test/Control×100 (% T/C) values, with T and C representing the fluorescence read-outs for wells with and without test compound, respectively, i.e. T/C represents the ratio of viable cell numbers following incubation of cell line with and without test compound. Based on the T/C values, relative IC₅₀ values were determined by non-linear regression (log [conc. of inhibitor] versus response (% T/C)) using the GraphPad Prism® analysis software (Prism 5 for Windows, version 5.01, GraphPad Software Inc., CA). For calculation of mean IC₅₀ values over the 40 cell lines as tested, the geometric mean was selected. If Graph Pad Prism could not calculate reliable IC₅₀ values by non-linear regression, the IC₅₀ value was estimated by visual inspection of the concentration-effect curve.

For mode-of-action analysis (MoAs), a Compare Analysis was performed. The individual IC₅₀ values of the test compound as obtained in 40 cell lines in the monolayer assay were correlated by a Spearman rank test to the corresponding IC₅₀ values for 94 standard agents as determined for these 40 cell lines. These standard agents represent the main MoAs of approved and experimental anti-cancer drugs. Similarities between the activity pattern of a test compound and those of standard drugs are expressed quantitatively as Spearman correlation coefficients. High correlations (ρ>0.6, p<0.05) between the activity patterns of two compounds are indicative of a similar MoAs.

In vitro antitumor activity of compounds in a panel of 40 human tumor cell lines:

Compound of formula (1) showed concentration-dependent activity in all cell lines as tested, i.e. cell lines derived from bladder, colon, gastric, head & neck, liver, lung (NSCLC), mammary, ovarian, pancreatic, prostate, renal and uterus cancer, as well as melanoma, pleuramesothelioma and sarcoma (Table 1). For all the 40 cell lines an IC₅₀<0.33 μM was achieved. The overall very strong antitumor potency was evident from a mean IC₅₀ value of 0.089 μM. The compound displayed a remarkable level of tumor selectivity (Table 2). Cell lines derived from bladder (2 out of 3 tested bladder cancer cell lines showed above-average sensitivity), colon (3/4), lung (5/6) and pancreatic cancer (3/3) were particularly sensitive. Mainly the strong selective activity towards the 5/6 cell lines derived from lung carcinoma (individual IC₅₀<0.05 μM) and towards the 3/3 pancreatic tumor cell lines (individual IC₅₀<0.06 μM) is to be highlighted. The most sensitive cell lines towards compound of formula (1) were found to be the pancreatic cancer cell line PAXF 546L (IC₅₀=0.016 μM) and the lung cancer cell line LXFA 526L (IC₅₀=0.021 μM). Compare Analysis revealed no significant correlations to any of the reference compounds (Spearman correlation coefficient ρ<0.4) indicating that the mode-of-action of compound of formula (1) is not represented by the standard agents used for Compare Analysis. The results are indicated in Table 1 and Table 2.

TABLE 1
In vitro anti-tumor activity of compound of formula
(1) in a panel of 40 human tumor cell lines

| | | | IC₅₀ |
|---|---|---|---|
| Sr No. | Cell line | Histotype | [μM] |
| 1 | BXF 1218L | Bladder | 0.046 |
| 2 | BXF 1352L | Bladder | 0.103 |
| 3 | BXF T 24 | Bladder | 0.050 |
| 4 | CXF 269L | Colon | 0.055 |
| 5 | CXF HCT 116 | Colon | 0.048 |
| 6 | CXF HT 29 | Colon | 0.137 |
| 7 | CXF RKO | Colon | 0.074 |
| 8 | GXF 251 L | Gastric | 0.093 |
| 9 | HNXF CAL 27 | Head and Neck | 0.039 |
| 10 | LIXF 575L | Liver | 0.295 |
| 11 | LXFA 289L | Lung | 0.041 |
| 12 | LXFA 526L | Lung | 0.021 |
| 13 | LXFA 629L | Lung | 0.040 |
| 14 | LXFL 1121L | Lung | 0.047 |
| 15 | LXFL 529L | Lung | 0.050 |
| 16 | LXFL H 460 | Lung | 0.146 |
| 17 | MAXF 401NL | Mammary | 0.064 |
| 18 | MAXF MCF 7 | Mammary | 0.276 |
| 19 | MAXF MDA 231 | Mammary | 0.090 |
| 20 | MEXF 1341L | Melanoma | 0.102 |
| 21 | MEXF 276L | Melanoma | 0.090 |
| 22 | MEXF 462NL | Melanoma | 0.149 |
| 23 | OVXF 899L | Ovarian | 0.301 |
| 24 | OVXF OVCAR3 | Ovarian | 0.195 |
| 25 | PAXF 1657L | Pancreas | 0.039 |
| 26 | PAXF 546L | Pancreas | 0.016 |
| 27 | PAXF PANC 1 | Pancreas | 0.059 |
| 28 | PRXF 22RV1 | Prostate | 0.159 |
| 29 | PRXF DU 145 | Prostate | 0.052 |
| 30 | PRXF LNCAP | Prostate | 0.122 |
| 31 | PRXF PC3M | Prostate | 0.047 |
| 32 | PXF 1118L | Pleuramesothelioma | 0.054 |
| 33 | PXF 1752L | Pleuramesothelioma | 0.187 |
| 34 | PXF 698L | Pleuramesothelioma | 0.178 |
| 35 | RXF 1781L | Renal | 0.260 |
| 36 | RXF 393NL | Renal | 0.323 |
| 37 | RXF 486L | Renal | 0.159 |
| 38 | SXF SAOS2 | Sarcoma | 0.185 |
| 39 | SXF TE671 | Sarcoma | 0.082 |
| 40 | UXF 1138L | Uterus | 0.126 |
| | Mean | | 0.089 |

TABLE 2
In vitro anti-tumor activity of compound of formula (1)
towards cell lines derived from selected tumor histotypes.
Tumor selectivity towards selected tumor histotypes¹⁾

| Mean IC₅₀ [μM] | Bladder | Colon | Lung | Breast | Melanoma |
|---|---|---|---|---|---|
| 0.089 | 2/3 | 3/4 | 5/6 | 1/3 | 0/3 |

| Mean IC₅₀ [μM] | Pancreas | Prostate | Pleurameso | Kidney |
|---|---|---|---|---|
| 0.089 | 3/3 | 2/4 | 1/3 | 0/3 |

¹⁾Number of cell lines among the respective histotype with individual IC₅₀ < mean IC₅₀

### CONCLUSION

Compound of formula (1) showed concentration-dependent antitumor activity (mean IC₅₀=0.089 μM) in a panel of 40 human tumor cell lines, which were derived from 15 different tumor histotypes.

1. A compound of formula (1), 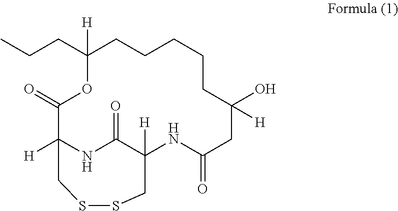 [SMILES: CCCC1CCCCCC(O)CC(=O)NC2CSSCC(NC2=O)C(=O)O1]
or an isomer or a tautomer or a mixture thereof; or a pharmaceutically acceptable salt thereof.

2. The compound of formula (1) as claimed in claim 1 characterised by:
(a) molecular weight of 418.16,
(b) molecular formula C₁₈H₃₀N₂O₅S₂,
(c) IR (KBr) spectrum 3345, 1646, 1671, 1715, 1459, 1399 cm⁻¹,
(d) ¹H NMR spectrum (500 MHz, DMSO-d₆): δ 8.8 (bs, 1H), 8.1 (bs, 1H), 4.9 (bs, 1H), 4.8 (bs, 1H), 4.5 (bs, 1H), 4.0 (s, 1H), 3.9 (s, 1H), 3.6 (m, 2H), 3.2 (m, 1H), 3.1 (d, 1H), 1.6-1.0 (m, 16H, methylene groups), 0.8 (t, 3H) (also depicted in FIG. 1), and
(e) ¹³C NMR spectrum (75 MHz, DMSO-d6): δ 174.17, 173.91, 169.42, 74.50, 70.30, 52.07, 51.26, 43.25, 42.10, 36.20, 35.60, 34.30, 32.90, 27.90, 26.70, 22.00, 18.30, 13.70.

3. A process for the production of the compound of formula (1) as claimed in claim 1, comprising the steps of:
(a) cultivating the microorganism belonging to Endophytic fungal strain (PM0509732/MTCC5544) or one of its variants or mutants under submerged aerobic conditions in a nutrient medium containing sources of carbon and nitrogen to produce the compound of formula (1),
(b) isolating the compound of formula (1) from the fermented broth, and
(c) purifying the compound of formula (1).

4. The process as claimed in claim 3, further comprising the step of converting the compound of formula (1) to its pharmaceutically acceptable salt.

5. A pharmaceutical composition comprising a therapeutically effective amount of the compound of formula (1) as claimed in claim 1, or an isomer or a tautomer or a mixture thereof; or a pharmaceutically acceptable salt thereof, and a pharmaceutically acceptable carrier or diluent.

6. A method for the treatment of cancer comprising administering to a mammal in need thereof a therapeutically effective amount of the compound of formula (1) as claimed in claim 1 or a pharmaceutically acceptable salt thereof.

7. The method according to claim 6, wherein the cancer comprises: bladder cancer, breast cancer, colorectal cancer, endometrial cancer, gastric cancer, head & neck cancer, kidney cancer, melanoma, non-small-cell lung cancer, ovarian cancer, pancreatic cancer, prostate cancer, renal cancer, soft tissue sarcoma, esophageal cancer, cervical cancer of uterus, testicular cancer, germ cell cancer, thyroid cancer, glioblastoma, cerebellar astrocytoma, cerebral astrocytoma, ependymoma, medulloblastoma, neuroblastoma, retinoblastoma, pleuramesothelioma, supratentorial primitive neuroectodermal and pineal tumors, visual pathway and hypothalamic glioma, brain stem glioma, liver cancer, Ewing's sarcoma family of tumors, osteosarcoma, malignant fibrous histiocytoma of bone, rhabdomyosarcoma, skin cancer, small-cell lung cancer, Wilms' tumors, acute lymphoblastic leukemia, adult acute myeloid leukemia, chronic lymphocytic leukemia, chronic myeloid leukemia, Hodgkin's disease, non-Hodgkin's lymphoma, hairy cell leukemia, multiple myeloma and primary central nervous system lymphoma.

8. (canceled)

9. (canceled)

10. (canceled)
